(19) United States
(12) Patent Application Publication
(10) Pub. No.: US 2005/0031537 A1
(43) Pub. Date: Feb. 10, 2005

(54) Multifunctional COX-2 inhibitors

(76) Inventors: Andrew J. Dannenberg, New York, NY (US); Kotha Subbaramaiah, New York, NY (US)

Correspondence Address: BACON & THOMAS, PLLC, 625 SLATERS LANE, FOURTH FLOOR, ALEXANDRIA, VA, 22314, US

(21) Appl. No.: 10/614,795

(22) Filed: Jul. 9, 2003

Related U.S. Application Data
(60) Provisional application No. 60/404,911, filed on Aug. 22, 2002.

(51) Int. Cl. A61K 49/00; A61K 31/415

(52) U.S. Cl. 424/9.2; 514/406

(57) ABSTRACT

Selective inhibitors of COX-2 and selective inhibitors of COX-1 are screened for COX protein independent therapeutic activity. Compounds passing screening testing are indicated for treatment of those having or at risk for cancer, Alzheimer's disease and atherosclerosis.

### CROSS-REFERENCE TO RELATED APPLICATIONS

This application claims the benefit of U.S. Provisional Application No. 60/404,911, filed Aug. 22, 2002.

### TECHNICAL FIELD

This invention is directed at a method for screening selective inhibitors of cyclooxygenase-2 (COX-2) for therapeutic functionality in addition to COX-2 protein inhibition and to use of multifunctional COX-2 inhibitors for treating patients having or at risk for cancer, Alzheimer's disease, or atherosclerosis.

### BACKGROUND OF THE INVENTION

It is known that high concentrations of nonsteroidal anti-inflamrnatory drugs (NSAIDs) can inhibit cell proliferation or induce apoptosis by COX-independent mechanisms. However, there is great debate as to whether these effects are clinically relevant because of the high concentrations of drug that are required. So far, NSAIDs are not approved for this purpose. Moreover, little is known about COX-independent effects of selective inhibitors of COX-2 at concentrations of drug that are believed to be clinically relevant.

### SUMMARY OF THE INVENTION

In a first embodiment, the invention herein is directed at a method of screening a selective inhibitor of COX-2 for functionality in addition to COX-2 protein inhibition, comprising screening for at least one COX protein inhibition independent therapeutic utility, e.g., comprising screening the selective inhibitor of COX-2 for at least one of, preferably for at least two of (a) activation of PPRE luciferase by at least 100%, (b) at least 50% decrease in level of or 50% downregulation of expression of Class I family of receptors tyrosine kinase, (c) at least 50% downregulation of expression of cyclin D1, (d) at least 50% downregulation of expression of HPV16 oncoproteins E6 and E7, (e) at least 50% increase in expression of PTEN, (f) at least 50% inhibition of tcf/lef/β-catenin-mediated promoter activation, and (g) at least 50% increase in expression of Nrf-2.

In a second embodiment, the invention is directed at a method for treating a patient having or at risk for cancer, Alzheimer's disease or atherosclerosis comprising administering a therapeutically effective amount of a selective inhibitor of COX-2 that meets at least two of (a), (b), (c), (d), (e), (f) and (g) of the screening method of the first embodiment. Preferably the administration in a therapeutically effective amount comprises administering the selective inhibitor of COX-2 in a dosage that not only inhibits COX-2 but also provides a function selected from the group consisting of activating PPRE luciferase by at least 100%, decreasing level of or downregulating expression of Class I family of receptors tyrosine kinase by at least 50%, downregulating expression of cyclin D1 by at least 50%, downregulating expression of HPV16 oncoproteins E6 and E7 by at least 50%, increasing expression of PTEN by at least 50%, inhibiting tcf/lef/β-catenin-mediated promoter activation by at least 50%, and inducing Nrf-2 by at least 50%.

In a third embodiment that overlaps the second embodiment, the invention is directed at a method for treating a patient having or at risk for cancer, Alzheimer's disease or atherosclerosis comprising administering a therapeutically effective amount of a selective inhibitor of COX-2 or a selective inhibitor of cyclooxygenase-1 (COX-1) that activates PPRE luciferase by at least 100%.

The term “selective inhibitor of cyclooxygenase-2” is used herein to mean compound which selectively inhibits cyclooxygenase-2 in preference to cyclooxygenase-1 and particularly compound for which the ratio of the IC₅₀ concentration (concentration inhibiting 50% of activity) for cyclooxygenase-1 to the IC₅₀ concentration for cyclooxygenase-2 is greater than 1. Such ratio is readily determined by assaying for cyclooxygenase-2 activity and assaying for cyclooxygenase-1 activity by the method set forth at column 39, line 55—column 40, line 36 of Talley et al. U.S. Pat. No. 5,633,272, which is incorporated herein by reference, and from the resulting data obtaining a ratio of IC₅₀s.

The term “selective inhibitor of cyclooxygenase-1” is used herein to mean compound which selectively inhibits cyclooxygenase-1 in preference to cyclooxygenase-2 and particularly compound for which the ratio of the IC₅₀ concentration (concentration inhibiting 50% of activity) for cyclooxygenase-2 to the IC₅₀ concentration for cyclooxygenase-1 is greater than 1. Such ratio is readily determined by assaying for cyclooxygenase-1 activity and assaying for cyclooxygenase-2 activity by the method set forth at column 39, line 55—column 40, line 36 of Talley et al. U.S. Pat. No. 5,633,272, which is incorporated herein by reference, and from the resulting data obtaining a ratio of IC₅₀s.

The term “COX protein inhibition independent therapeutic activity” is used herein to mean therapeutic activity unrelated to the inhibition of prostaglandin synthesis.

### BRIEF DESCRIPTION OF THE DRAWINGS

FIG. 1 depicts bar graphs of concentration versus PPRE luciferase activity and compares PPRE luciferase activity for the selective inhibitors of COX-2 SC-236, SC-58125 and PTPBS to that of ciglitazone and shows results of Example III.

FIG. 2 depicts bar graphs of concentration versus PPRE luciferase activity and compares PPRE luciferase activity for the selective inhibitors of COX-2 N-(3-pyridyl)-indomethacin amide (denoted indomethacin amide) and indomethacin heptyl ester (denoted indomethacin heptyl) to that of the NSAID indomethacin and shows results of Example III.

FIG. 3 depicts bar graphs of concentration versus PPRE luciferase activity for the selective inhibitor of COX-1 SC-560 and shows results of Example III.

### DETAILED DESCRIPTION

We turn now to the first embodiment of the invention herein which is directed at a method of screening a selective inhibitor of COX-2 for functionality in addition to COX-2 protein inhibition, comprising screening for at least one COX protein inhibition independent therapeutic utility.

This method preferably comprises screening the selective inhibitor of COX-2 for at least one of, very preferably for at least two of (a) activation of PPRE luciferase by at least 100% (i.e., at least doubling of luciferase activity based on data that have been normalized with β-galactosidase activity), (b) at least 50% decrease in level of or 50% downregulation of expression of Class I family of receptors tyrosine kinase, (c) at least 50% downregulation of expression of cyclin D1, (d) at least 50% downregulation of expression of HPV16 oncoproteins E6 and E7, (e) at least 50% increase in expression of PTEN, (f) at least 50% inhibition of tcf/lef/62 -catenin-mediated promoter activation, and (g) at least 50% increase in expression of Nrf-2.

We turn now to criterion (a), that is activation of PPRE luciferase by at least 100%. PPRE-luciferase refers to a DNA construct containing the peroxisome proliferator activated receptor (PPAR) binding element joined to luciferase. Cells are transfected with PPRE-luciferase DNA. Ligands of PPAR induce luciferase activity. The ability of a test compound to stimulate PPRE-luciferase signifies that the test compound activates PPAR-mediated gene transcription. PPRE means peroxisome proliferator response element and the method by which PPRE-luciferase is constructed is described in Schoonjans, K., et al, J. Biol. Chem. 270, No. 33, 19269-19276 (Aug. 18, 1995). Use of PPRE-luciferase in a transient transfection to measure increase in promoter activity is disclosed in Subbaramaiah, K. et al, J. Biol. Chem. 270, No. 15, 12440-12448 (Apr. 13, 2001).

A specific test used in examples herein for screening selective inhibitors of COX-2 and selective inhibitors of COX-1 for activation of PPRE luciferase by at least 100% follows:

Cells were seeded at a density of 5×10⁴ cells/well in 6-well dishes and grown to 50-60% confluence. For each well, 1.8 μg of PPRE3-tk-luciferase construct plasmid DNA (described in Forman, B. M., et al, Cell 83, 803 (1995)) plus 0.2 μg of pSV-β-galactosidase were introduced into cells using 8 μg of LipofectAMINE as per the manufacturer's instructions (Invitrogen, CA). After 7 h of incubation, the medium was replaced with basal medium. After transfection, cells were treated with test compound for 24 h. Reporter activities which include luciferase and β-galactosidase were measured in cellular extract 24 h later. Six wells were used for each of the conditions. Luciferase activity represents data that have been normalized with β-galactosidase.

The luciferase assay was performed as per the BD Pharmingen assay reagents. The Enhanced Luciferase Assay Kit provides a firefly luciferase substrate formulation (Substrates A and B) and cell lysis buffer intended for use in measuring luciferase expressed by transfected cells. Media from cell culture plates was removed and rinsed twice with phosphate buffered saline. Enough 1X cell lysis buffer was added to cover cells and incubated at room temperature for 15-20 min. Cells were dislodged by scraping and transferred to microcentrifuge tubes. Cells were spun for 5-10 sec to remove cellular debris. 20-100 μl cell extract was placed into an assay cuvette. One automatic injector type luminometer is used for assay. Manually 100 μl of Substrate A was added to the assay cuvette and automatically 100 μl of Substrate B was injected. Measurements were performed using a luminometer, for a measurement time of 10 seconds.

The β-galactosidase assay was performed as follows:

50 μl of cell lysate from above was incubated with 50 μl of o-nitrophenyl-β-D-galactoside (ONPG; 4 mg/mL) in Z buffer, (Na₂HPO₄.7H₂O (0.06M), NaH₂PO₄.H₂O (0.04M), 1M KCl (0.01M), 1M MgSO₄ (0.001M), β-mercaptoethanol (BME) (0.05M) pH 7.0). The reaction was stopped after sufficient yellow color has developed. The yellow color developed is measured at A420 nM.

Cell lines successfully used were 184B5, 184B5/HER, HCA7, HCT116, SKBR3 and BT474.

We turn now to the criterion (b), that is at least 50% decrease in level of or 50% downregulation of expression of Class I family of receptors tyrosine kinase. The Class I family of receptors tyrosine kinase is described in Reese, M. D., et al Stem Cells 15, pages 1-8 (1997), the whole of which in incorporated herein by reference. Members of the family include HER-2/neu, HER-3, HER-4 and epidermal growth factor receptor (EGFR) and are single-chain membrane spanning proteins which have significant homology to one another including about 80% amino acid identity in the tyrosine kinase domain.

HER-2/neu (erbB-2) gene product is a 185-kDA transmembrane receptor tyrosine kinase that is described in some detail in said Reese et al publication and overexpression thereof has been associated with tumor growth in several kinds of cancer.

Epidermal growth factor receptor (EGFR) is a 170 kDA glycoprotein. It is a prototypical transmembrane protein that consists of an extracellular ligand-binding domain, a transmembrane domain, and an intracellular domain that possesses intrinsic tyrosine kinase activity. After ligand binding, EGFR undergoes dimerization which is essential for activation of its enzymatic kinase activity. EGFR is thus autophospliorylated and transphosphorylated on tyrosine residues, and the phosphorylated residues become the site of association of effector proteins. Overexpressed EGFR is intimately involved in modulating the epidermal growth factor growth signal and is considered as likely to confer a growth advantage. This conclusion is supported by the observation that tumor growth in nude mice is inhibited by treatment with anti-EGFR antibodies and tumorigenicity in nude mice is inhibited through blockage of the tyrosine kinase activity of the receptor. EGFR has been found to be overexpressed in many malignancies.

To evaluate the expression of Class I family of receptors tyrosine kinase (EGFR, HER-2/neu), Western blotting can be performed. Antibodies for EGFR and HER-2/neu can be obtained from Santa Cruz Biotechnology, Inc.

Specific tests used in examples herein for screening selective inhibitors of COX-2 for causing at least 50% decrease in level of or 50% downregulation of expression of Class I family of receptors tyrosine kinase, follows:

Expression of EGFR (performed in 184B5, 184B5/HER, 1483, LNCaP, HeLa and CaSki cells)and HER-2/neu (performed in 184B5/HER, SKBR3 and BT474 cells) was assessed in cells after treatment with test compound for 24 hours. 5×10⁶ cells were used for the treatment. Western blotting technique was used to assess the expression levels in cells treated with the test compound compared to vehicle alone. Western blotting was carried out as follows:

Cell lysates were prepared by treating cells with lysis buffer (150 mM NaCl, 100 mM Tris (pH 8.0), 1% Tween 20, 50 mM diethyldithiocarbamate, 1 mM EDTA, 1 mM phenylmethylsulfonyl fluoride, 10 μg/ml aprotinin, 10 μg/ml trypsin inhibitor and 10 μg/ml leupeptin). Lysates were sonicated for 20 s on ice and centrifuged at 10,000×g for 10 min to sediment the particulate material. The protein concentration of the supernatant was measured by the method of Lowry, O. H., et al., J. Biol. Chem. 193, 265-275 (195 1). SDS/PAGE was performed under reducing conditions on 10% polyacrylamide gels as described by Laemmli, U.K., Nature (Lond.) 227, 680-685 (1970). The resolved proteins were transferred onto nitrocellulose sheets as detailed by Towbin, H. et al., Proc. Natl. Acad. Sci. USA 76, 4350-4354 (1979). The nitrocellulose membrane was then incubated with anti-EGFR antiserum or anti-HER-2/neu antiserum. Secondary antibody to IgG conjugated to horseradish peroxidase was used. The various antisera were purchased from Santa Cruz Biotechnology, Inc., CA. The blots were probed with the Renaissance Western blot detection system according to the manufacturer's instructions (PerkinElmer Life Sciences, Boston, Mass.).

We turn now to the criterion (c), that is at least 50% downregulation of expression of cyclin D1.

Cyclin D1 is a protein that is important in regulating cell proliferation and is described in Ortega, S., et al, Biochimica et Biophysica Acta 1602, 73-87, (2002), the whole of which is incorporated by reference.

A specific test used in examples herein for screening selective inhibitors of COX-2 for causing at least 50% downregulation of expression of cyclin D1, follows:

Effects on expression of cyclin D1 was investigated in 184B5, 184B5/HER, HCA7, HCT116, SKBR3 and BT474 cells. The expression was assessed in cells after treatment with test compound or vehicle using 5×10⁴ cells for 24 hours. Western blotting technique was used as follows to assess the expression levels of cyclin D1 in cells treated with the test compound and compared to levels in cells treated with vehicle alone. Cell lysates were prepared by treating cells with lysis buffer as described above. SDS/PAGE was performed under reducing conditions on 10% polyacrylamide gels as above. The resolved proteins were transferred onto nitrocellulose sheets. The nitrocellulose membrane was then incubated with anti-cyclin D1 antiserum. Secondary antibody to IgG conjugated to horseradish peroxidase was used. The various antisera were purchased from Santa Cruz Biotechnology, Inc., CA. The blots were probed with the Renaissance Western blot detection system according to the manufacturer's instructions (PerkinElmer Life Sciences, Boston, Mass.).

We turn now to the criterion (d), that is at least 50% downregulation of expression of HPV16 oncoproteins E6 and E7.

HPV means human papillomavirus and proteins E6 and E7 expressed by HPV16 have been found to be involved in cervical displasia and invasive cervical cancer and other types of cancers as well. Protein E6 and E7 which are expressed by HPV16 are believed to play a major role in carcinogenesis.

A specific test used in examples herein for screening selective inhibitors of COX-2 for causing at least 50% downregulation of HPV 16 oncoproteins E6 and E7, follows:

Western blotting and Northern blotting techniques were used to evaluate the expression of HPV E6 and E7 proteins. The cell lines used for the study were CaSKi and SiHa. The cells were treated with the test compound and cell lysate was obtained as described above and subjected to Western blotting analysis. The Western blotting analysis was performed as described above except anti-HPV 16 E7 antibody was used to probe the blot. The antibody was obtained from Santa Cruz Biotechnology, Inc. CA. For Northern blotting, total cellular RNA was isolated from cell monolayers using an RNA isolation kit from QIAGEN Inc. after treatment with the test compound. 10 μg of total cellular RNA per lane were electrophoresed in a formaldehyde-containing 1.2% agarose gel and transferred to nylon-supported membranes. After baking, membranes were prehybridized overnight in a solution containing 50% formamide, 5×sodium chloride-sodium phosphate-EDTA buffer (SSPE), 5×Denhardt's solution, 0.1% SDS and 100 μg/ml single-stranded salmon sperm DNA and then hybridized for 12 h at 42° C. with radiolabeled cDNA probes for human HPV16 E6 or E7 cDNAs and 18S rRNA. E6 and E7 (as described in Woodworth, C. D., Cancer Res. 16, 4397-4402 (2000)) and 18S rRNA probes were labeled with [³²P]-CTP by random priming. After hybridization, membranes were washed twice for 20 min at room temperature in 2×SSPE-0.1% SDS, twice for 20 min in the same solution at 55° C., and twice for 20 min in 0.1×SSPE-0.1% SDS at 55° C. Washed membranes were then subjected to autoradiography.

We turn now to criterion (e), i.e., at least 50% increase in expression of PTEN.

PTEN is a tumor suppressor that inhibits cell proliferation and induces programmed cell death. PTEN (also know as MMAC-1 or TEP-1) is described in Yamada, K. M., et al, Journal of Cell Science 114, 2375-2382, the whole of which is incorporated herein by reference.

A specific test used in examples herein for screening selective inhibitors of COX-2 for causing at least 50% increase in expression of PTEN, follows:

Western blotting and Northern blotting techniques were used to evaluate the expression of PTEN. The expression of PTEN was evaluated in HCA7, HCT116, SKBR3, 184B5/HER, 184B5 and BT474 cells. 5×10⁶cells were used to treat either vehicle alone or the test compound. The Western blotting analysis was performed as described above except anti-PTEN antibody was used to probe the blot. The antibody was obtained from Santa Cruz Biotechnology, Inc. CA. Northern blotting for PTEN was performed as described above and cDNA for PTEN as described in Tamura, M., et al, Science 5; 280 (5369):1614-1617 (1998), was used as a probe.

We turn now to criterion (f), i.e., at least 50% inhibition of tcf/lef/β-catenin-mediated promoter activation.

Tcf/lef/β-catenin is a transcription factor complex that has been implicated in the regulation of genes involved in carcinogenesis. Increased tcf/lef/β-catenin-mediated gene expression has been linked to pathogenesis of colon cancer. Tcf/lef/β-catenin is described in Barker, N., et al, Adv. Cancer Res. 77, 1-24 (2000), the whole of which is incorporated herein by reference.

A specific test used in Example II herein for screening selective inhibitors of COX-2 for causing at least 50% inhibition of tcf/lef/β-catenin-mediated promoter activation, follows.

Cells (cell lines were 184B5, 184B5/HER, HCA7, HCT116, SKBR3 and BT474) were split into 6 well dishes and the next day were transiently transfected using 8 μg of LipofectAMlNE as per the manufacturer's instructions (Invitrogen, CA). 1.8 μg/dish of the wildtype Tcf/Lef reporter plasmid TOP flash or the mutant plasmid FOP flash was transfected into the cells and 0.2 μg of pSV-β-galactosidase. FOPflash differs from TOPflash by the mutation of its Tcf binding sites and serves to differentiate Tef/-catenin-mediated signaling from background (Upstate Biotechnology, Inc.). Cells were incubated overnight and were treated for 24 hours with either vehicle or test compound. Luciferase activity in the extracts was measured as described above and was corrected for background by subtraction of FOP-FLASH values from corresponding TOP-FLASH values.

We turn now to criterion (g), i.e. at least 50% increase in expression of Nrf-2. Nrf-2 is a transcription factor that regulates the expression of genes involved in xenobiotic metabolism and is described in Ramos-Gomez, M., et al, PNAS98, No. 5, 310-3415 Mar. 13, 2001).

A specific test used in Example II herein for screening selective inhibitors of COX-2 for causing at least 50% increase in expression of Nrf-2, follows:

Western blotting and Northern blotting techniques were used to evaluate the expression of Nrf-2. The expression of Nrf-2 was evaluated in HCA7, HCT116, SKBR3, CaSki, 184B5/HER, 184B5 and BT474 cells. 5×10⁶cells were used to treat either vehicle alone or the test compound. The Western blotting analysis was performed as described above except anti-Nrf-2 antibody was used to probe the blot. The antibody was obtained from Santa Cruz Biotechnology, Inc. CA. Northern blotting for Nrf-2 was performed as described above and cDNA for Nrf-2 as described in Gong, P., et al., J.Biol Chem 20; 276:27018-27025 (2001) was used as a probe.

Cell lines recited above are 184B5, 184B5/HER, HCA7, 1483, BT474, SKBR3, SiHa, CaSki, LNCaP, HeLa and HCT116. Of these, all are tumerigenic except for 184B5.

The 184B5 cell line is an immortalized human breast epithelial cell line that was established from a reduction mammoplasty and is described in Stamfer, M. R., et al., Proc. Natl. Acad Sci. USA 82, 2394-2398 (1985).

The 184B5/HER cell line is described in Pierce, J. H., et al., Oncogene 6, 1189-1194 (1991) and was derived from stably transfecting 184B5 cells with a mutationally activated HER-2/neu oncogene; these cells form rapidly growning tumors when injected into athymic nude mice.

The HCA7 cell line is described in Marsh, K. A., et al, J. Pathol. 170, 441-450 (1993).

The 1483 cell line is described in Sacks, P. G., et al, Cancer Res. 48, 2858-2866 (1988).

The BT474 cell line is a human breast adenocarcinoma cell line which overexpresses HER-2/neu and was obtained from the American Type Culture Collection (Manassas, Va.) and bears accession number ATCC HTB-20.

The SKBR3 cell line was obtained from the American Type Culture Collection (Manassas, Va.) and bears accession number ATCC HTB-30.

The SiHa cell line was obtained from the American Type Culture Collection (Manassas, Va.) and bears accession number ATCC HTB-35.

The CaSki cell line is a prototypic cervical cancer cell line known to be infected with HPV 16 and was obtained from the American Type Culture Collection (Manassas, Va.) and bears accession number ATCC CRL-1550.

The LNCaP cell line was obtained from the American Type Culture Collection (Manassas, Va.) and bears accession number ATCC CRL-1740.

The HeLa cell line was obtained from the American Type Culture Collection (Manassas, Va.) and bears accession number ATCC CCL-2.

The HCT116 cell line was obtained from the American Type Culture Collection (Manassas, Va.) and bears accession number ATCC CCL-247.

Passing of one or more screening tests of the first embodiment of the invention herein maximizes the opportunity of the agent passing the test, being successful for the treatment of and in the second embodiment herein. The more of the tests (a), (b), (c), (d), (e), (f) and (g) passed, the greater the likelihood of success.

We turn now to the second embodiment of the invention herein which is directed at a method for treating a patient having or at risk for cancer, Alzheimer's disease or atherosclerosis, comprising administering a therapeutically effective amount of a selective inhibitor of COX-2 that meets at least one of, preferably at least two of, (a), (b), (c), (d), (e), (f) and (g).

The cancers to which the second embodiment applies are all cancers and include cancers of the bladder, breast, cervix, colorectum, including colon, skin, esophagus, head and neck, lung including non small-cell lung cancers, kidney, pancreas and prostate, and endometrial cancers, gastric cancers, gliomas, gall bladder, bile duct, hepatocellular carcinomas, ovarian cancers and salivary cancers. Conditions to which the second embodiment applies where the patient is at risk for cancer include oral premalignant lesions, cervical intraepithelial neoplasia, chronic hepatitis, bile duct hyperplasia, atypical adenomatous hyperplasia of lung, prostatic intraepithelial neoplasia, bladder dysplasia, actinic keratoses of skin, colorectal adenomas, gastric metaplasia, and Barrett's esophagus.

For the selective inhibitors of COX-2 for the second embodiment, the ratio of the IC₅₀ concentration for COX-1 to the IC₅₀ concentration for COX-2 is preferably greater than 5, very preferably greater than 100. Selective inhibitors of COX-2 that meet at least two of (a), (b), (c), (d), (e), (f) and (g) include diaryl heterocycles that comprise the moiety (1) described hereinafter including those with the structure (2) hereinafter where R₁ is C₁-C₆ alkyl, halogen or H as described hereinafter and R₂ is sulfonamide or methyl sulfone. Particular compounds meeting the structure (2) where R₁ is C₁-C₆ alkyl, halogen or H and R₂ is sulfonamide or methyl sulfone that have been found to meet at least two of (a), (b), (c), (d), (e), (f) and (g), include celecoxib, SC-236, PTPBS and SC-58125. Other selective inhibitors of COX-2 that have been found to meet at least two of (a), (b), (c), (d), (e), (f) and (g) include indomethacin analogs including N-(3-pyridyl)-indomethacin amide, indomethacin heptyl ester; indomethacin ester, 4-methoxyphenyl-; N-(4-acetamidophenyl)-indomethacin amide; N-(2-phenylethyl)-indomethacin amide; and indomethacin N-octylamide. The chemical structures and/or nomenclature for these are set forth below in the description of the third embodiment of the invention. These compounds are all commercially available except that celecoxib can be purified from capsules of Celebrex™ sold for patient care. Selective inhibitors of COX-2 that are believed to meet at least two of (a), (b), (c), (d), (e), (f) and (g) include diaryl heterocycles comprising the structures (3) hereinafter or (4) hereinafter including refecoxib and valdecoxib or are found among the selective inhibitors of COX-2 listed or described in WO 00/13685, the whole of which is incorporated herein by reference.

The dosage for the selective inhibitor of COX-2 for the second embodiment is a therapeutically effective amount (that ameliorates symptoms and/or pathology of cancer, Alzheimer's disease, atherosclerosis and/or prevents or slows the occurrence or progression thereof) that not only inhibits COX-2 (but not COX-1) but also provides a function selected from the group consisting of activating PPRE luciferase by at least 100%, decreasing level of or downregulating expression of Class I family of receptors tyrosine kinase by at least 50%, downregulating expression of cyclin D1 by at least 50%, downregulating expression of HPV16 oncoproteins E6 and E7 by at least 50%, increasing expression of PTEN by at least 50%, inhibiting tcf/lef/β-catenin-mediated promoter activation by at least 50%, and increasing expression of NrF-2 at least 50%. For example, while the recommended dose for celecoxib for artlritis is 100-200 mg bid and for familial adenomatous polyposis (FAP) is 400 mg bid, 600 mg bid might be required to target (a), (b), (c), (d), (e), (f) and/or (g). In general, the dosage ranges from 0.1 to 30 mg/kg with the dosage for any particular agent varying within the range. Routes of administration include oral, intravenous and topical.

We turn now to the third embodiment of the invention herein which is directed at a method for treating a patient having or at risk for cancer, Alzheimer's disease or atherosclerosis, comprising administering a therapeutically effective amount of a selective inhibitor of COX-2 or a selective inhibitor of COX-1 that activates PPRE luciferase by at least 100%. The test for activation of PPRE luciferase by at least 100% is test (a) of the first embodiment.

The cancers to which the third embodiment applies are all cancers and include those cancers listed above for the second embodiment and the precancerous conditions listed above for the second embodiment.

For the selective inhibitors of COX-2 for the third embodiment, the ratio of the lC₅₀ concentration for COX-1 to the IC₅₀ concentration for COX-2 is preferably greater than 5, very preferably greater than 100.

Selective inhibitors of COX-2 that have been found to activate PPRE luciferase by at least 100% include indomethacin derivatives including N-(3-pyridyl)-indomethacin amide (N-3PIA), Cayman Chemical 70274, which has the structure 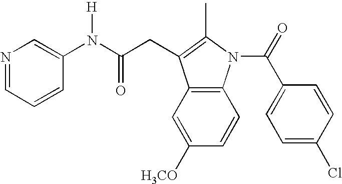
indomethacin heptyl ester (Cayman Chemical 70271) which has the structure 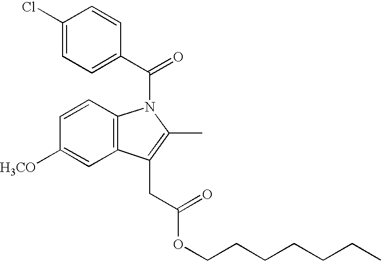
N-(2-phenylethyl)-indomethacin amide (N-2PIA), Cayman Chemical 70272, which has the structure 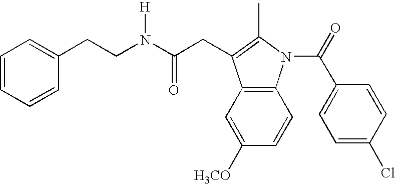
indomethacin ester, 4-methoxyphenyl-(Calbiochem 405271), also known as 1-(p-chlorobenzoyl)-5-methoxy-2-methyl-1H-indole-3-acetic acid, 4-methoxyphenyl ester, which has the structure 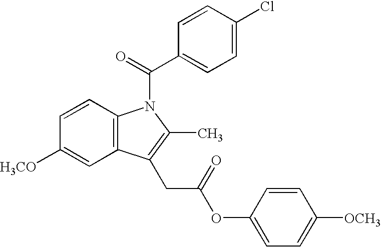
N-(4-acetamidophenyl-indomethacin amide (n-4-AcetIA), Cayman Chemical 70278, which has the structure 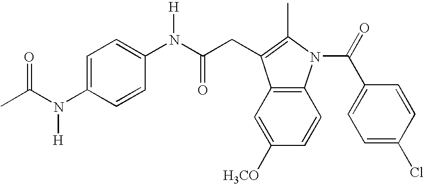
and indomethacin amide, N-octyl, Calbiochem K405270, which is also called 1-(p-chlorobenzoyl)-5-methoxy-2-methyl-1H-indole-3-octylacetamide and has the structure

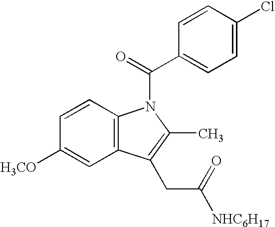

The above-identified indomethacin derivatives are commercially available.

Other selective inhibitors of COX-2, and selective inhibitors of COX-1, that have been found to activate PPRE luciferase by at least 100% are diaryl heterocycles, e.g., comprising the moiety:

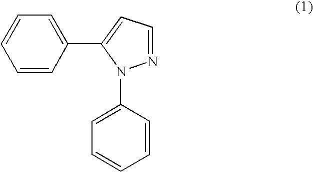

One group of these diaryl heterocycles has the structure 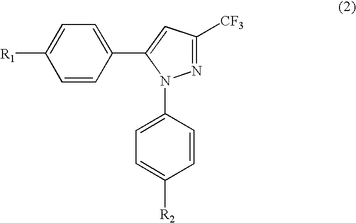
where R₁ is C₁-C₆alkyl, e.g., methyl, halogen, e.g., chlorine or fluorine, or H, and R₂ is sulfonamide, i.e., SO₂NH₂, methyl sulfone or OR where R is C₁-C₆alkyl, e.g., methoxy. Compounds of structure (2) which are selective inhibitors of cyclooxygenase-2 and have been found to activate PPRE by at least 100% include 4-5[-(4-methylphenyl)-3-trifluoromethyl)-1H-pyrazol-1-yl]benzenesulfonamide, also known as celecoxib, which is sold under the trade name Celebrex™; 4-[5-(4-chlorophenyl)-3-(trifluoromethyl)-1H-pyrazol-1-yl]benzenesulfonamide, also known as SC-236, Calbiochem 56505; 4-[5-phenyl-3-(trifluoromethyl)-1H-pyrazol-1-yl]benezenesulfonamide, also known as PTPBS; and the compound of structure (2) where R₁ is fluorine and R₂ is methylsuflonyl which may be described as 4-[5-(4-fluorophenyl)-3-(trifluoromethyl)-1H-pyrazol-1-yl]benzenemethylsulfone, also known as SC-58125, Cayman Chemical 70655. Compounds of the structure (2) where R₂ is OR include selective inhibitors of cyclooxygenase-1. A compound of structure (2) where R₂ is OCH₃ and is a selective inhibitor of cyclooxygenase-1 that has been found to activate PPRE by at least 100% has the structure (2) where R₁ is chlorine and R₂ is OCH₃ and is named 5-(4-chlorophenyl)-1-(4-methoxyphenyl)-3-(trifluoromethyl)-1H-pyrazole and is also know as SC-560, Cayman Chemical 70340. The compounds specifically named in this paragraph are commercially available except that celecoxib may be purified from Celebrex™ capsules sold for patient care.

Selective inhibitors of COX-2 that are believed to activate PPRE luciferase by at least 100% include diaryl heterocycles comprising the moiety 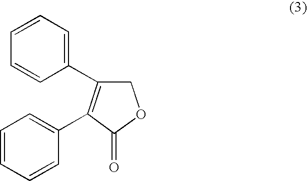
for example, refecoxib, and diaryl heterocycles having the moiety 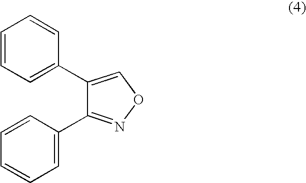
for example, valdecoxib.

Other selective inhibitors of COX-2 that are believed to activate PPRE luciferase by at least 100% are found among the selective inhibitors of COX-2 listed or described in WO 00/13685, the whole of which is incorporated herein by reference.

The dosage for the selective inhibitors of COX-2 for the third embodiment is a therapeutically effective amount (that ameliorates symptoms and/or pathology of cancer, Alzheimer's disease or atherosclerosis and/or prevents or slows the occurrence or progression thereof) that not only inhibits COX-2 but also provides the function of activating PPRE by at least 100% and in general ranges from 0.1 to 30 mg/kg with the dosage for any particular agent varying within the range. The dosage for the selective inhibitors of COX-1 for the third embodiment is a therapeutically effective amount (that ameliorates symptoms and/or pathology of cancer, Alzheimer's disease or atherosclerosis and/or prevents or slows the occurrence or progression thereof) that also provides the function of activating PPRE by at least 100%; in general the dosage ranges from 0.1 to 30 mg/kg with the dosage for any particular agent varying within the range. Routes of administration include oral, intravenous and topical.

Variations of the second and third embodiments exclude administering selective inhibitor of COX-2 to treat HPV16 mediated cancer and/or cancers associated with the overexpression of HER-2/neu and/or exclude administering celecoxib (Celebrex™).

### EXAMPLE I

Tests utilized were those specifically described above.

SC-236 was incubated with 5×10⁶ 184B5 cells in a 10 cm diameter dish. Incubations were carried out for 24 hours. Transient transfections were performed utilizing a PPRE-luciferase construct. SC-236 caused dose-dependent activation of luciferase activity. The highest concentration of SC-236 led to 150% activation of the promoter. Western blotting was performed for EGFR, cyclin D1 and PTEN. SC-236 caused dose-dependent decreases in levels of EGFR and cyclin D1 and dose-dependent increase in level of PTEN. Levels of EGFR and cyclin D1 decreased by more than 50% following treatment with SC-236. By contrast, SC-236 caused more than a 100% increase in amounts of PTEN.

SC-236 (0-5.0 μM) was incubated with 3×10⁶ CaSki cells (prototypic cervical cancer cells known to be infected with HPV16). Incubations were carried out for 24 hours. Western blotting was carried out to determine levels of HPV16 E7 protein. The Western blot results showed that SC-236 caused dose-dependent suppression of E7 protein. At the high concentration of SC-236, greater than 50% inhibition was observed. Northern blotting was carried out and showed dose-dependent decreases in E6 mRNA and more than a 50% decrease in amounts of E6 mRNA, following treatment with SC-236.

SC-236 has a ratio of the IC₅₀ concentration for COX-1 to the IC₅₀ concentration for COX-2 of 1780:1 and as shown above, passes screening tests (a), (b), (c), (d) and (e) and therefore is a likely candidate for utility for treating a patient having or at risk for cancer, AlzIheimer's disease or atherosclerosis.

### EXAMPLE II

A table indicating which tests (a) through (g) have been carried out and the results of the tests for celecoxib, SC-236, SC-58125, PTPBS and the six indomethacin derivatives specifically named above and SC-560 is set forth below. The tests (a)-(g) utilized are those specifically described above. The cell lines used that gave a positive result for test (a) were 184B5, 184B5/HER, HCA7, HCT116, SKBR3 and BT474; for test (b) for HER-2/neu were 184B5/HER, BT474 and SKBR3; for test (b) for EGFR were 184B5, 184B5/HER, 1483, LNCaP, HeLa and CaSki; for test (c) were 184B5, 184B5/HER; for test (d) were CaSki and SiHa; for test (e), were 184B5, 184B5/HER, HCA7, HCT116, SKBR3 and BT474; for test (f) were 184B5, 184B5/HER, HCA7, HCT116, SKBR3 and BT474; and for test (g) were 184B5, 184B5/HER, HCA7, HCT116, SKBR3, BT474 and CaSki. The indomethacin analogs designated in the table as 1, 2, 3, 4, 5 and 6, respectively, were indomethacin heptyl ester; N-(2-phenylethyl)-indomethacin amide; indomethacin ester, 4-methoxyphenyl; N-(3-pyridyl)-indomethacin amide, N-(4-acetamidophenyl-indomethacin amide; and indomethacin N-octylamide. A plus sign in the table indicates a positive result, that is meeting the test for each cell line as indicated above as providing a positive result.

The table follows:

TABLE I

| | | | | | Indomethacin Analogs | | | | | | |
|---|---|---|---|---|---|---|---|---|---|---|---|
| Test | Celecoxib | SC-236 | SC-58125 | PTPBS | 1 | 2 | 3 | 4 | 5 | 6 | SC-560 |
| (a) | + | + | + | + | + | + | + | + | + | + | + |
| (b) | + | + | + | + | + | + | + | | | | + |
| HER- | | | | | | | | | | | |
| 2/neu | | | | | | | | | | | |
| (b) | + | + | + | + | + | | + | | + | | + |
| EGFR | | | | | | | | | | | |
| (c) | + | + | + | + | | + | | | + | | |
| (d) | + | + | + | + | + | + | | + | | + | + |
| (e) | + | + | + | + | + | | | + | | | + |
| (f) | + | + | + | + | + | | + | + | | | + |
| (g) | + | + | + | + | + | | + | | + | | + |

All the compounds specifically mentioned above, meet at least two of the tests (a)-(g).

### EXAMPLE III

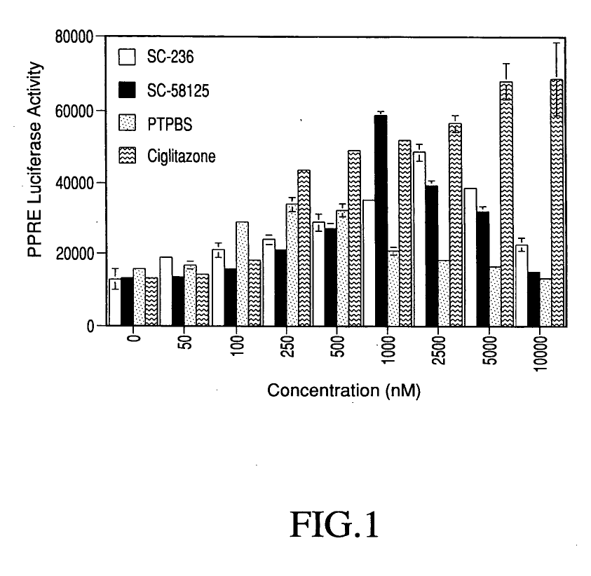

Tests were carried out on SC-236, SC-58125, PTPBS, ciglitazone, N-(3-pyridyl)-indomethacin amide, indomethacin heptyl ester, indomethacin and SC-560. These compounds were tested for increase of PPRE luciferase by the method particularly described in conjunction with test (a) for the first embodiment, at concentrations set forth in FIG. 1-3.

FIG. 1 depicts results for SC-236, SC-58125 and PTPBS in comparison to ciglitazone. Each of the selective inhibitors of COX-2 causes dose-dependent activation of PPRE luciferase and activates PPRE luciferase by more than 100% at concentrations tested. The selective inhibitors of COX-2 are much more active at concentrations tested than NSAIDs tested at the same concentrations.

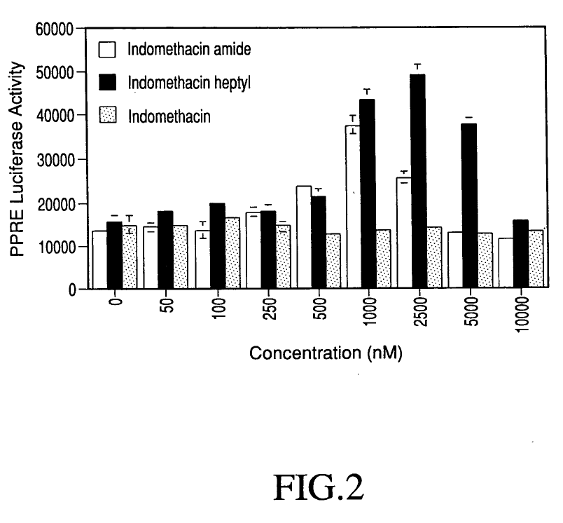

FIG. 2 depicts results for N-(3-pyridyl)-indomethacin amide and indomethacin heptyl ester in comparison to indomethacin. The results show that converting the indocmethacin to a selective inhibitor of COX-2 results in a better PPAR agonist and that the COX-2 inhibitors cause dose dependent activation of PPRE luciferase and activate PPRE luciferase by more than 100% at concentrations tested.

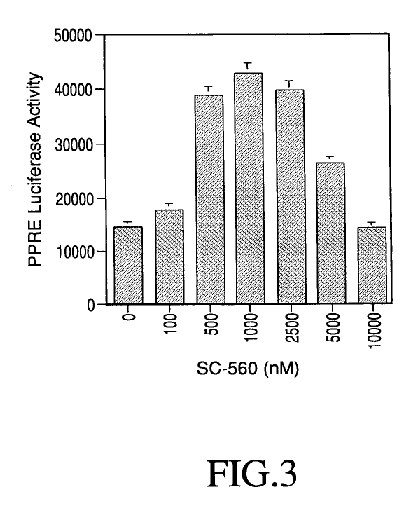

FIG. 3 depicts results for SC-560 and shows it causes dose dependent activation of PPRE luciferase and activates PPRE luciferase by more than 100% at concentrations tested.

### EXAMPLE IV

A 55 year old male presents with symptoms of constipation and rectal bleeding and is diagnosed with colon cancer. The patient is given and maintained on celecoxib or SC-236, 800 mg bid, and the rate of progression of the colon cancer decreases.

### EXAMPLE V

A 77 year old female presents with symptoms of memory loss and is diagnosed with Alzheimer's disease. The patient is given and maintained on a 200 mg bid dose of indomethacin heptyl ester. The progression of the symptoms stops.

### EXAMPLE VI

A 62 year old female patient presents with symptoms of chest discomfort on exertion and is diagnosed with atherosclerosis. The patient is given and maintained on a 200 mg bid dose of SC-560. The progression of the atherosclerosis stops.

### EXAMPLE VII

A 62 year old male presents with a white patch on his tongue and is diagnosed as having an oral premalignant lesion. The patient is given lozenges containing 100 mg of SC-236 twice a day for six months with resolution of the premalignant lesion.

### EXAMPLE VIII

A 37 year old woman presents to her gynecologist for evaluation. A routine pap test reveals highly atypical squamous cells. On biopsy, the patient is found to have cervical intraepithelial neoplasia. HPV 16 is detected. Treatment with 0.5% SC-236 in a cream applied to a sponge/cervical cap is begun and given for six months. At the end of six months, the patient undergoes repeat biopsy and there is no further evidence of cervical intraepithelial neoplasia. The SC-236 is discontinued with no recurrence of cervical intraepithelial neoplasia.

When SC-560 is substituted for SC-236, there is no evidence of cervical intraepithehal neoplasia and no recurrence after discontinuation of the drug.

Variations

Many variations of the above will be obvious to those skilled in the art. Therefore, the invention is defined by the claims.

1. A method for screening a selective inhibitor of COX-2 for functionality in addition to COX-2 protein inhibition, comprising screening for at least one COX protein inhibition independent therapeutic activity.

2. The method of screening of claim 1 which comprises screening the selective inhibitor of COX-2 for at least two of (a) activation of PPRE luciferase by at least 100%, (b) at least 50% decrease in level of or 50% downregulation of expression of Class I family of receptors tyrosine kinase, (c) at least 50% downregulation of expression of cyclin D1, (d) at least 50% downregulation of expression of HPV16 oncoproteins E6 and E7, (e) at least 50% increase in expression of PTEN, (f) at least 50% inhibition of tcf/lef/β-catenin-mediated promoter activation, and (g) at least 50% increase in level of Nrf-2.

3. A method for treating a patient having or at risk for cancer, Alzheimer's disease or atherosclerosis comprising administering a therapeutically effective amount of selective inhibitor of COX-2 that meets at least two of (a), (b), (c), (d), (e), (f) and (g).

4. A method for treating a patient having or at risk for cancer, Alzheimer's disease or atherosclerosis comprising administering a therapeutically effective amount of selective inhibitor of COX-2 or selective inhibitor of COX-1 that activates PPRE luciferase by at least 100%.

5. A method for treating a patient having or at risk for cancer, Alzheimer's disease or atherosclerosis, comprising administering a selective inhibitor of COX-2 in a dosage that not only inhibits COX-2 but also which provides a fimction selected for the group consisting of activating PPRE luciferase by at least 100%, decreasing level of or downregulating expression of Class I family of receptors tyrosine kinase by at least 50%, downregulating expression of cyclin D1 by at least 50%, downregulating expression of HPV16 oncoproteins E6 and E7 by at least 50%, increasing expression of PTEN by at least 50%, inhibiting tcf/lef/β-catenin-mediated promoter activation by at least 50% and inducing Nrf-2 by at least 50%.
